SEQUENCE LISTING (5 sequences)

SEQ ID NO: 1 - DNA, 22 bp, Artificial Sequence
  Synthetic oligonucleotide
ccctggacactggactatgatg

SEQ ID NO: 2 - RNA, 22 nt, Artificial Sequence
  Synthetic oligonucleotide
caucauaguccaguguccaggg

SEQ ID NO: 3 - DNA, 22 bp, Artificial Sequence
  Synthetic oligonucleotide
catcatagtccaatgtccaggg

SEQ ID NO: 4 - DNA, 22 bp, Artificial Sequence
  Synthetic oligonucleotide
cataataatccaatgtcaagag

SEQ ID NO: 5 - RNA, 22 nt, Artificial Sequence
  Synthetic oligonucleotide
uaacacuucaucggucccguag

(19) United States
(12) Patent Application Publication
(10) Pub. No.: US 2010/0193357 A1
(43) Pub. Date: Aug. 5, 2010

(54) QUANTITATIVE ANALYSIS METHOD FOR MICRORNAS

(76) Inventors: Po-Ling CHANG, Xinzhuang City (TW); Yu-Sun CHANG, Linkou Shiang (TW); Shu-Jen CHEN, Taipei City (TW); Hua-Chien CHEN, Taipei City (TW)

Correspondence Address: Muncy, Geissler, Olds & Lowe, PLLC, 4000 Legato Road, Suite 310, FAIRFAX, VA, 22033, US

(21) Appl. No.: 12/363,133

(22) Filed: Jan. 30, 2009

(51) Int. Cl. G01N 27/26; G01N 33/00

(52) U.S. Cl. 204/453; 436/94

(57) ABSTRACT

The present invention discloses a quantitative analysis method for microRNAs, wherein a fluorescence-labeled DNA probe, which is equinumerous and completely complementary to a microRNA, hybridize with the microRNA. The products of hybridization include the fluorescence-labeled DNA probe containing 22 nucleotides and the probe-microRNA duplex containing 22 base pairs. The products of hybridization is introduced into a capillary by the pressure difference between two ends of the capillary and the siphon effect and separated by electrophoresis. A laser is used to induce fluorescence from the products of hybridization. Then, the intensities of fluorescence are measured and analyzed.

### BACKGROUND OF THE INVENTION

1. Field of the Invention

The present invention relates to a quantitative analysis method for microRNAs, particularly to a quantitative analysis method for microRNAs, wherein a fluorescence-labeled nucleic acid probe, an in-capillary electrophoresis and a laser-induced fluorescence are used to directly detect a microRNA without using any nucleic acid amplification process.

2. Description of the Related Art

The microRNA (miRNA)-regulated physiological mechanism has been a hot subject for recent years. MicroRNA was found in nematoids as long as 14 years ago. However, the processed microRNA has only 21-23 bases and has a short life. Therefore, researchers almost neglected the role microRNAs play in biological bodies during the past more than ten years. Via the methodology of biological information, the scholars of MIT estimated that the genes of human beings have more than 300 microRNAs, and that the expressions of over one third genes are regulated by microRNAs. The research team of Deepak Srivastava found that if the microRNA—miR-1-2, which expresses specifically in the muscle of the heart, is culled out from the genes of the mouse embryos, the embryos of mice will develop to have a congenital cardionosis—ventricular septal defect, and the conduction of the cardiac nerves is also affected. The research team of Baofeng Yang and Zhiguo Wang in Harbin Medical University found that the overexpression of miR-1-2 will result in cardiac arrhythmia in mature mice. The two research results show the importance of miR-1-2 in the development and physiological regulation of the heart. In the paper, the research team of Deepak Srivastava not only firstly established the mode of culling out microRNA genes from mice but also proposed an auxiliary method predicting the probability of the combination of microRNA and the genes regulated thereby according to the free energy. Later, other two papers sequentially found that miR-133 and miR208 play roles in the pathological mechanism of cardiac hypertrophy. The abovementioned research results are sufficient to show that the small but powerful microRNAs play important roles not only in cardiac development but also in cardiac diseases.

According to the existing documents, microRNAs regulate or cancerate cells via inhibiting the growth of mRNAs or proteins. There are also documents pointing out that microRNAs have tissue specificity, and that the expressions of most of microRNAs in cancer tissues are distinct from that in normal tissues. Using the conventional gene microarray technology to analyze the mRNAs is hard to distinguish the cancer cells from the normal cells. Therefore, the hospitals can only use pathological section examinations to determine whether there are cancer cells. However, analyzing the expressions of microRNAs not only can distinguish cancer cells from normal cells but also can identify the types of cancer cells. It is expected that the expression distribution of microRNAs will be used in pathological analyses to aid identifying the cancer status.

At present invention, the mainstream technologies for testing microRNAs include the microarray chip method and the RT-qPCR (Reverse Transcription-quantitative Polymerase Chain Reaction) method. The microarray chip method can detect several types of microRNAs simultaneously. However, the microarray chip method is limited by the price and reproducibility thereof and thus hard to popularize. The RT-qPCR method has a high sensitivity. However, the experimental error is also amplified, which decreases the accuracy of the quantitative analysis.

### SUMMARY OF THE INVENTION

The primary objective of the present invention is to provide a quantitative analysis method for microRNAs, wherein a fluorescence-labeled nucleic acid probe, an in-capillary electrophoresis and a laser-induced fluorescence are used to directly detect a microRNA without using any nucleic acid amplification process, whereby the conventional problems are solved essentially.

The present invention proposes a quantitative analysis method for microRNAs, which comprises steps: providing a sample reagent having a plurality of unamplified equilength nucleic acid molecules; mixing the sample reagent and a probe, wherein the probe is a fluorescence-labeled polynucleotide, and the nucleotide sequence of the probe is completely complementary to the microRNA of the sample reagent; hybridizing the sample reagent and the probe; separating the products of hybridization; using a laser to induce fluorescence from the separated products and detecting the intensities of fluorescence.

Below, the embodiments are described in detail in cooperation with the drawings to make easily understood the objectives, characteristics and functions of the present invention.

### BRIEF DESCRIPTION OF THE DRAWINGS

FIG. 1 is a flowchart of a quantitative analysis method for microRNAs according to the present invention;

FIG. 2 is a diagram showing the analyses of the melting curves of the mixture solutions;

FIGS. 3A-3C are diagrams schematically showing the hybridization process according to the present invention;

FIGS. 4A-4C are diagrams comparing the intensities of fluorescence; and

FIG. 5 is a diagram showing comparing the intensities of fluorescence after extraction.

### DETAILED DESCRIPTION OF THE INVENTION

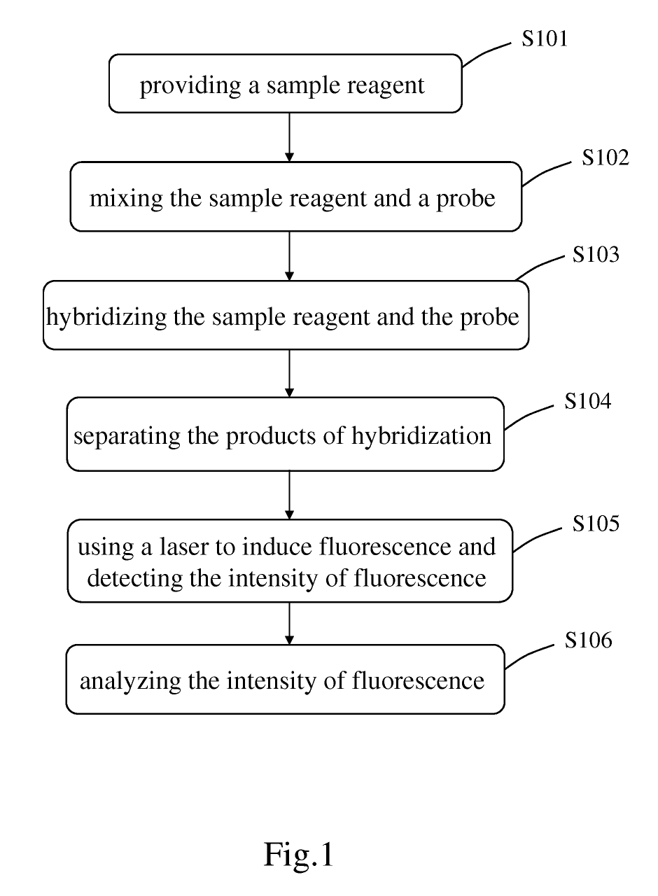

Refer to FIG. 1 a flowchart of a quantitative analysis method for microRNAs according to the present invention.

In Step S101, a sample reagent is provided, and the sample reagent has a plurality of unamplified and equilength nucleic acid molecules, wherein the nucleic acid molecules are ribonucleic acid molecules, deoxyribonucleic acid molecules, or the mixture of them, and the sample reagent contains a target microRNA, and the sequence of the target microRNA can encode a portion of the EBV (Epstein-Barr Virus) genome.

In Step S102, the sample reagent is mixed with a probe. The probe is a fluorescence-labeled polynucleotide, and the nucleotide sequence of the probe is completely complementary to that of the microRNA of the sample reagent. The probe may be a commercial synthetic high-sensitivity fluorescence-labeled single-strand ribonucleic acid, such as Alexa Fluor® 532. The probe is equinumerous to the microRNA of the sample reagent. In other words, the probe and the microRNA of the sample reagent have the same number of nucleotides.

Refer to Table.1 showing oligonucleotide sequences and the most stable duplex between the probe and the targets of the sample reagent.

TABLE 1

| Seq. | | |
|---|---|---|
| I.D. No. | Name and sequence | Most stable duplexes |
| 1 | BART7-AS (dye-labeled antisense DNA probe) | |
| | 5′-Alexa Fluor* | |
| | 532-CCCTGGACACTGGACTATGATG-3′ | |
| 2 | BART7-SE (BART7 miRNA) | BART7-AS + BART7-SE ΔG = −39.4 kcal/mol |
| | 5′-CAUCAUAGUCCAGUGUCCAGGG-3′ | 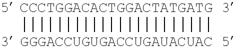 |
| 3 | BART7 1-nt | BART7-AS + BART7 1-nt ΔG = −18.9 kcal/mol |
| | 5′-CATCATAGTCCAATGTCCAGGG-3′ | 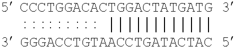 |
| 4 | BART7 5-nt | BART7-AS + BART7 5-nt ΔG = −3.4 kcal/mol |
| | 5′-CATAATAATCCAATGTCAAGAG-3′ | 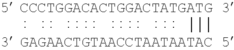 |
| 5 | BART9-SE | BART7-AS + BART9-SE ΔG = −8.4 kcal/mol |
| | 5′-UAACACUUCAUCGGUCCCGUAG-3′ | 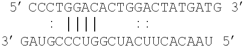 |

For example, the sample reagent contains a target microRNA BART7-SE with a serial number of 2, and the probe contains the single-strand ribonucleic acid BART7-AS with a serial number of 1.

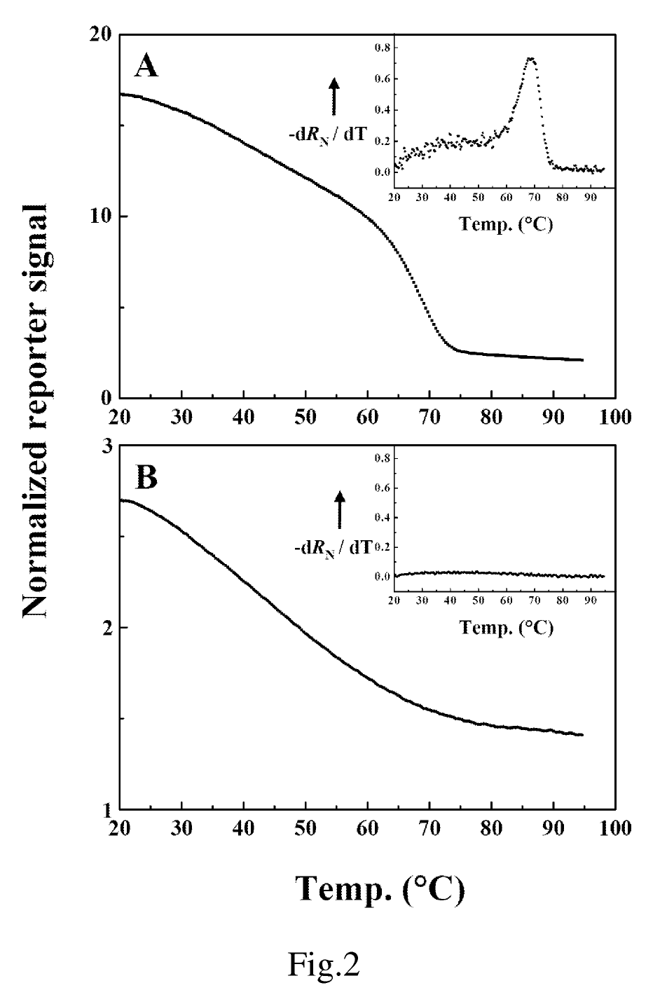

A cationic surfactant (such as a cationic detergent) is added into the mixture solution to accelerate the hybridization reaction between the probe and the microRNA, whereby the hybridization reaction can occur at a temperature much lower than the theoretical melting temperature, and whereby the hybridization reaction between the probe and the microRNA needn't be optimized by the melting temperature. In other words, even though the melting temperatures of two microRNAs have a difference of 15° C., they can still hybridize at an identical temperature in the present invention. In this embodiment, 0.1 mM of cationic surfactant—SSC-buffered CTAB (sodium sesquicitrate-buffered cetyltrimethylammonium bromide) is used as the buffer solution. Refer to FIG. 2 for the analyses of the melting curves of the mixture solutions, wherein Solution A uses 0.5 mM of sodium sesquicitrate as the buffer solution, and Solution B uses the abovementioned SSC-buffered CTAB as the buffer solution. The normalized reporter signal (Rn) can be calculated from the intensity of the fluorescence described in the succeeding steps.

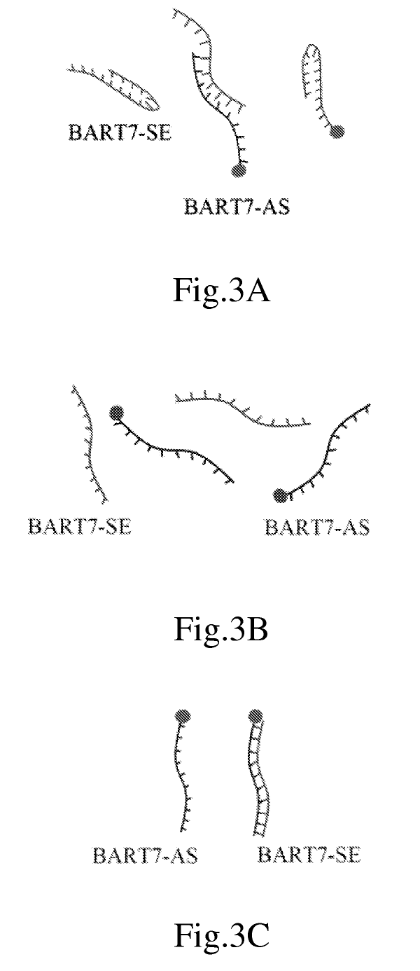

In Step S103, the hybridization is undertaken; the nucleic acid probe and the sample reagent are mixed evenly and denatured by heating. The mixture solution in an initial state (shown in FIG. 3A) is heated to a temperature of 95° C. and maintained at the temperature for 5 minutes, whereby the tested solution containing the probe and the sample reagent is denatured (shown in FIG. 3B). Next, the tested solution is cooled down to a temperature of 50° C. and maintained at the temperature for 10 minutes, whereby the nucleic acid and the microRNA are renatured in a 10 μL reaction solution (shown in FIG. 3C). Thus is completed hybridization. Then, 1 μL of 10 mM of an anionic surfactant—SDS (sodium dodecyl sulfate) is added to neutralize CATB lest the positively-charged CATB adheres to the wall of the capillary.

In principle, the hybridization temperature is lower than the theoretical melting temperature. As the nucleotide sequence of the probe is completely complementary to the nucleotide sequence of the microRNA of the sample reagent, the products of hybridization include the fluorescence-labeled DNA probe (free probe containing 22 nucleotides) and the probe-microRNA duplex containing 22 base pairs.

In Step S104, the products of hybridization are separated. The hybridized mixture solution is guided into a capillary with a hydrodynamic injection. The two ends of the capillary are inserted into a buffer solution containing high-concentration urea and a linear polymer, such as a buffer solution containing 7M of urea and 2% high-molecular weight (8,000,000 g/mole) poly(ethylene oxide). Next, a high-voltage (such as 10 KV) current is applied to the capillary to induce electrophoresis, whereby the urea and the poly(ethylene oxide) move to the negative electrode, and the products move to the positive electrode. The high-concentration urea can maintain the structure of the single-strand DNA probe and prevent the hybridization-generated two-strand nucleic acid structure from being denatured, whereby the single-strand DNA probe and the hybridization-generated two-strand nucleic acid structure can be separated in the capillary. Via electrophoresis, a high-concentration high-viscosity linear polymer can be introduced into the capillary for a high-resolution nucleic acid separation without using a high-pressure pump. Further, the capillary is soaked in a solution of sodium hydroxide to generate an electroosmotic flow in electrophoresis.

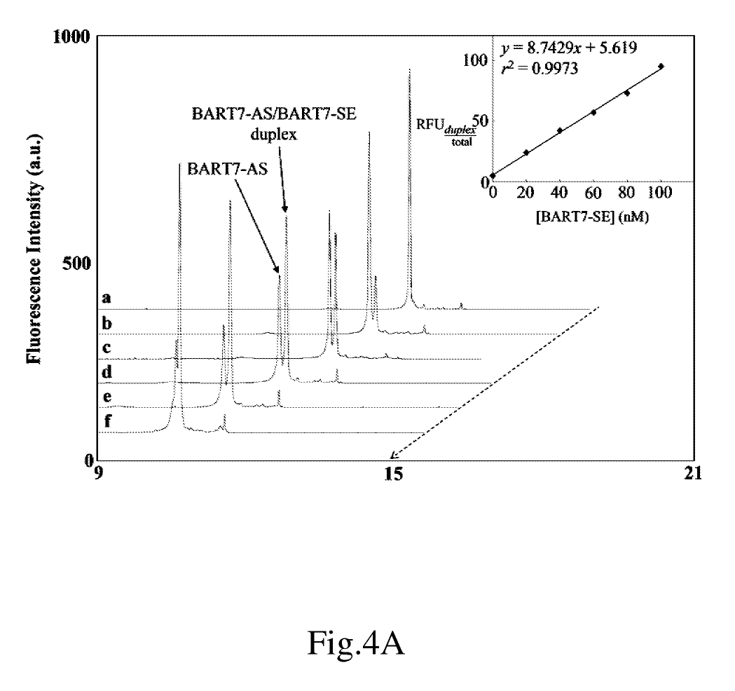

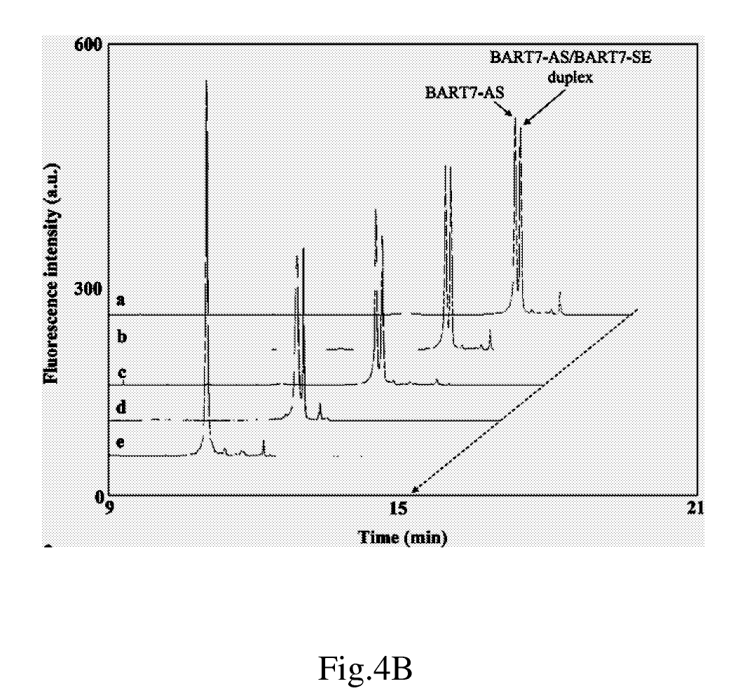

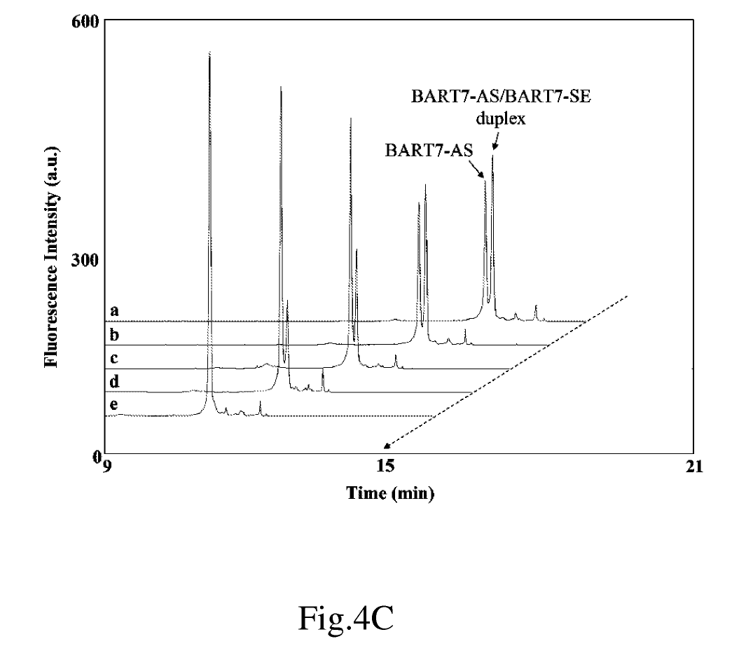

In Step S105, a laser is used to induce fluorescence. Refer to FIGS. 4A-4C diagrams showing the intensities of fluorescence. FIG. 4A shows the results of a test undertaken in a capillary with a length of 40 cm and an effective length of 33 cm and in the presence of an electric field intensity of 250 V/cm. In hybridization, the concentration of BART7-AS is 10 nM, and the concentrations of BART7-SE are respectively 0 M (Curve (a)), 2 nM (Curve (b)), 4 nM (Curve (c)), 6 nM (Curve (d)), 8 nM (Curve (e)), and 10 nM (Curve (f)). FIG. 4B shows the test results of specificity, wherein various potential interference molecules are respectively spiked into the tested solutions. The concentrations of BART7-AS and BART7-SE are 10 nM and 5 nM respectively. Various potential interference materials were added into the hybridization vials, wherein Curve (a) is an interference-free case, Curve (b) a case with the total RNA (2 μg) extracted from a nasopharyngeal cancer cell line—HK-1 cells, Curve (c) a case with BART9 miRNA (10 μM) added, Curve (d) a case with BART7 DNA in 5 nt mismatch (10 μM) added, and Curve (e) a case with BART7 DNA in 1 nt mismatch (10 μM) added. FIG. 4C shows the results of tolerance tests, wherein various concentrations of BART7 DNA in 1 nt mismatch are respectively added into the tested solution. The concentrations of BART7-AS and BART7-SE are 10 nM and 5 nM respectively. The interference materials are added to the reaction vials at the following concentrations: interference-free (Curve (a)), 10 nM (Curve (b)), 100 nM (Curve (c)), 1 μM (Curve (d)), and 10 μM (Curve (e)). Therefore, no false positive result is detected even in the presence of a 2000-fold excess of a single nucleotide-mismatched interference material.

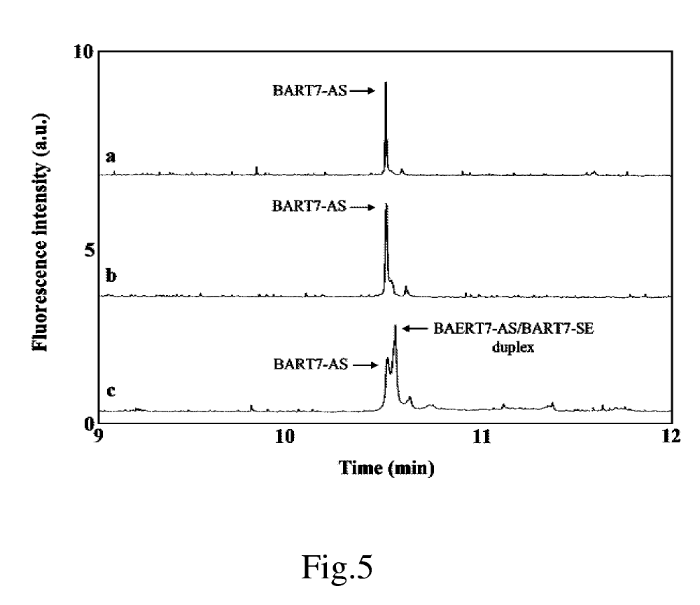

In Step S106, the intensities of fluorescence are analyzed. The intensity of fluorescence is continuously measured as a function of migration time. FIG. 5 shows the test results of a case with BART7-AS alone (a), a case with an RNA sample extracted from a nasopharyngeal cancer cell line (HK-1) (b), and a case with an RNA sample extracted from an EBV (Epstein-Barr Virus)-infected nasopharyngeal cancer cell line (C666-1) (c).

In conclusion, the present invention proposes a quantitative analysis method for microRNAs, which uses a cationic surfactant to accelerate the hybridization reaction of nucleic acids, whereby nucleic acids can hybridize at different temperatures without modifying the lengths of microRNAs or being optimized by the melting temperatures. In the presence of electrophoresis, the method of the present invention can introduce a high-concentration high-viscosity linear polymer into a capillary for a high-resolution separation of nucleic acids without using a high-pressure pump. Further, the capillary needs no cleaning after the analysis of the present invention.

Via the aid of high-concentration urea, the present invention not only can maintain the structure of the single-strand DNA probe and but also can prevent the hybridization-generated two-strand nucleic acid from being denatured and enable the separation of the reaction products in the capillary. In tests, when an interference agent is overdosed into the reactants, such as the total RNA of human beings, the microRNA of an unrelated EB virus or a single nucleotide-mismatched microRNA, the present invention does not output a false positive result. Therefore, the present invention has specificity higher than other existing methods. Even though the single nucleotide-mismatched microRNA has a concentration 2000 times higher than that of the target microRNA (BART7), none false positive peak is observed in the electrophoregram.

The embodiments described above are only to exemplify the present invention but not to limit the scope of the present invention. Any equivalent modification or variation according to the spirit of the present invention is to be also included within the scope of the present invention, which is based on the claims stated below.

What is claimed is:

1. A quantitative analysis method for microRNAs comprising steps:
providing a sample reagent having a plurality of unamplified equilength nucleic acid molecules;
mixing said sample reagent and a probe, wherein said probe is a fluorescence-labeled polynucleotide, and said probe has a molecular length identical to that of a microRNA (micro ribonucleic acid) of said sample reagent, and said probe has a nucleotide sequence completely complementary to that of said microRNA of said sample reagent;
performing a hybridization of said sample reagent and said probe;
separating products of said hybridization; and
using a laser to induce fluorescence from said products and measuring intensities of said fluorescence.

2. The quantitative analysis method for microRNAs according to claim 1 further comprising a step of analyzing said intensities of said fluorescence.

3. The quantitative analysis method for microRNAs according to claim 1, wherein said hybridization further comprises steps:
heating and denaturing a tested solution of said sample reagent and said probe; and
cooling said tested solution to renature said sample reagent and said probe and complete said hybridization.

4. The quantitative analysis method for microRNAs according to claim 3, wherein a cationic surfactant is added to said tested solution to accelerate hybridizing microRNAs of said sample reagent and said probe during said hybridization.

5. The quantitative analysis method for microRNAs according to claim 4, wherein said cationic surfactant is CATB (cetyltrimethylammonium bromide).

6. The quantitative analysis method for microRNAs according to claim 4, wherein said cationic surfactant makes said hybridization occur at a temperature much lower than a theoretical melting temperature.

7. The quantitative analysis method for microRNAs according to claim 4, wherein said cationic surfactant exempts said hybridization from being optimized by a theoretical melting temperature and enables two microRNAs having a melting-temperature difference of 15° C. to hybridize at an identical temperature simultaneously.

8. The quantitative analysis method for microRNAs according to claim 4, wherein an anionic surfactant is used to neutralize said cationic surfactant lest said cationic surfactant survive in succeeding steps.

9. The quantitative analysis method for microRNAs according to claim 8, wherein said anionic surfactant is SDS (Sodium Dodecyl Sulfate).

10. The quantitative analysis method for microRNAs according to claim 1, wherein said nucleic acid molecules of said sample reagent are selected from a group consisting of RNAs (Ribonucleic acids), DNAs (Deoxyribonucleic acids), and mixtures of RNAs and DNAs.

11. The quantitative analysis method for microRNAs according to claim 1, wherein said probe and said microRNA are equinumerous in nucleotides.

12. The quantitative analysis method for microRNAs according to claim 1, wherein each of said intensities of said fluorescence is continuously measured as a function of migration time.

13. The quantitative analysis method for microRNAs according to claim 1, wherein a sequence of said microRNA encodes a portion of an EBV (Epstein-Barr Virus) genome.

14. The quantitative analysis method for microRNAs according to claim 1, wherein said products of said hybridization include a fluorescence-labeled DNA probe and a duplex of said probe and said microRNA.

15. The quantitative analysis method for microRNAs according to claim 1, wherein said “separating products of said hybridization” further comprises steps:
injecting said products of said hybridization into a capillary placed in a buffer solution;
applying a current to said capillary to induce electrophoresis in said capillary;
maintaining said current for a predetermined interval of time; and
separating said products of said hybridization.

16. The quantitative analysis method for microRNAs according to claim 15, wherein said buffer solution includes a denaturant.

17. The quantitative analysis method for microRNAs according to claim 16, wherein said denaturant enables said probe to maintain a single-strand structure without damaging a hybridization-generated two-strand reaction product of said probe and said microRNA during said electrophoresis.

18. The quantitative analysis method for microRNAs according to claim 15, wherein said capillary is soaked in a solution of sodium hydroxide to generate an electroosmotic flow during said electrophoresis.
